(19) United States
(12) Patent Application Publication
(10) Pub. No.: US 2025/0108231 A1
(43) Pub. Date: Apr. 3, 2025

(54) Device for Disinfecting a Surface Using a Light Source

(71) Applicant: Becton, Dickinson and Company, Franklin Lakes, NJ (US)

(72) Inventors: Kevin M. Ryan, Whitehouse Station, NJ (US); Angela Marie Huenerfauth, Morris Plains, NJ (US); Shishir Prasad, Ramsey, NJ (US)

(21) Appl. No.: 18/687,849

(22) PCT Filed: Sep. 1, 2022
(86) PCT No.: PCT/US2022/042342

§ 371 Date: Feb. 29, 2024

Related U.S. Application Data
(60) Provisional application No. 63/240,019, filed on Sep. 2, 2021.

(51) Int. Cl. A61N 5/06

(52) U.S. Cl. CPC A61N 5/0624; A61N 5/0616; A61N 2005/0626; A61N 2005/065; A61N 2005/0663

(57) ABSTRACT

A disinfection device may include a housing having a concave shape, a light source coupled to the housing, where the light source provides light configured to disinfect a surface, a contact sensor coupled to the housing, where the contact sensor is configured to detect contact with a body of a user.

### CROSS-REFERENCE TO RELATED APPLICATION

The present application claims priority to U.S. Provisional Application Ser. No. 63/240,019, entitled “Device for Disinfecting a Surface Using a Light Source”, filed Sep. 2, 2021, the entire disclosure of which is hereby incorporated by reference in its' entirety.

### BACKGROUND

Intravenous (IV) therapy may refer to a medical technique that is used to deliver fluids, medications, and/or nutrition directly into a body of a patient via an IV line (e.g., an IV cannula, an IV catheter, etc.) inserted into a vein. IV therapy may be used for rehydration, to provide nutrition to a person who cannot consume food or water by mouth, and/or to administer medications or other medical therapy, such as blood products or electrolytes. In some instances, an IV injection site for IV therapy may require a disinfecting process. The disinfecting process may involve the use of a topical disinfectant following removal of an IV dressing at the IV injection site. For example, the disinfecting process for an IV injection site may be to remove an IV dressing covering the IV injection site and wiping down affected areas with a topical disinfectant, such as a wipe saturated with rubbing alcohol.

However, there may be significant drawbacks to removing the IV dressing that covers the IV injection site. For instance, removing the IV dressing may increase stress on the skin of a patient, which in many cases, in particular with elderly patients, may cause tearing and/or bruising of the skin. In addition, an IV line at the IV injection site may become dislodged from external or sub-dermal layers where the IV line enters into the skin.

Therefore, there is an increasing need for disinfecting IV injection sites, including IV catheter insertion sites, without disturbing the IV dressing that holds an IV line in place.

### SUMMARY

Accordingly, provided are improved systems, devices, products, apparatus, and/or methods for disinfecting a surface using a light source.

According to some non-limiting embodiments or aspects, provided is a disinfection device comprising a housing having a concave shape, a light source coupled to the housing, wherein the light source provides light configured to disinfect a surface, a contact sensor coupled to the housing, wherein the contact sensor is configured to detect contact with a body of a user.

According to some non-limiting embodiments or aspects, provided is a device comprising a housing having a concave shape, a printed circuit board (PCB) positioned within the housing, a light source attached to the PCB, wherein the light source provides light configured to disinfect a surface, a contact sensor coupled to the housing, wherein the contact sensor is configured to detect contact with a body of a user, and a proximity sensor attached to the PCB, wherein the proximity sensor is configured to detect whether the proximity sensor is in proximity to the body of the user.

According to some non-limiting embodiments or aspects, provided is a device comprising a housing having a concave shape, a printed circuit board (PCB) positioned within the housing, a light source attached to the PCB, wherein the light source provides light configured to disinfect a surface, and a contact sensor comprising a plurality of electrical traces and a sensor electrically coupled to the plurality of electrical traces, wherein the sensor is attached to the PCB, and wherein the contact sensor is configured to detect contact with a body of a user.

Further non-limiting embodiments or aspects are set forth in the following numbered clauses:

Clause 1: A disinfection device comprising: a housing having a concave shape; a light source coupled to the housing, wherein the light source provides light configured to disinfect a surface; a contact sensor coupled to the housing; and wherein the contact sensor is configured to detect contact with a body of a user.

Clause 2: The disinfection device of clause 1, wherein the light source provides light in a wavelength that is configured to disinfect a surface.

Clause 3: The disinfection device of clause 1 or 2, wherein the light source comprises at least one light emitting diode (LED).

Clause 4: The disinfection device of any of clauses 1-3, wherein the light source provides antimicrobial blue light (aBL).

Clause 5: The disinfection device of any of clauses 1-4, further comprising: a printed circuit board (PCB) positioned within the housing; and wherein the light source is attached to the PCB.

Clause 6: The disinfection device of any of clauses 1-5, wherein the contact sensor comprises a plurality of electrical traces.

Clause 7: The disinfection device of any of clauses 1-6, wherein the contact sensor comprises a sensor in electrical contact with the plurality of electrical traces.

Clause 8: The disinfection device of any of clauses 1-7, wherein the contact sensor comprises a first electrical trace and a second electrical trace, wherein the first electrical trace and the second electrical trace are separated by a gap.

Clause 9: The disinfection device of any of clauses 1-8, wherein the contact sensor is configured to detect contact with the body of the user based on a reduction in resistance between the first electrical trace and the second electrical trace when the first electrical trace and the second electrical trace are in contact with the body of the user.

Clause 10: The disinfection device of any of clauses 1-9, wherein the housing comprises a rim, and wherein the contact sensor is positioned on the rim of the housing.

Clause 11: The disinfection device of any of clauses 1-10, wherein the light source is configured to be activated based on the contact sensor detecting contact with the body of the user.

Clause 12: The disinfection device of any of clauses 1-11, further comprising: a band configured to hold the housing in contact with the body of the user.

Clause 13: A device comprising: a housing having a concave shape; a printed circuit board (PCB) positioned within the housing; a light source attached to the PCB, wherein the light source provides light configured to disinfect a surface; a contact sensor coupled to the housing, wherein the contact sensor is configured to detect contact with a body of a user; and a proximity sensor attached to the PCB, wherein the proximity sensor is configured to detect whether the proximity sensor is in proximity to the body of the user.

Clause 14: The device of clause 13, wherein the light source comprises at least one light emitting diode (LED) that provides light in a wavelength that is configured to disinfect a surface.

Clause 15: The device of clause 13 or 14, wherein the at least one LED provides antimicrobial blue light (aBL).

Clause 16: The device of any of clauses 13-15, wherein the contact sensor comprises a first electrical trace and a second electrical trace, wherein the first electrical trace and the second electrical trace are separated by a gap; and wherein the contact sensor is configured to detect contact with the body of the user based on a reduction in resistance between the first electrical trace and the second electrical trace when the first electrical trace and the second electrical trace are in contact with the body of the user.

Clause 17: A device comprising: a housing having a concave shape; a printed circuit board (PCB) positioned within the housing; a light source attached to the PCB, wherein the light source provides light configured to disinfect a surface; and a contact sensor comprising a plurality of electrical traces and a sensor electrically coupled to the plurality of electrical traces, wherein the sensor is attached to the PCB; and wherein the contact sensor is configured to detect contact with a body of a user.

Clause 18: The device of claim 17, wherein the light source comprises at least one light emitting diode (LED) that provides antimicrobial blue light (aBL).

Clause 19: The device of claim 17, wherein the contact sensor comprises a first electrical trace and a second electrical trace, wherein the first electrical trace and the second electrical trace are separated by a gap; and wherein the contact sensor is configured to detect contact with the body of the user based on a reduction in resistance between the first electrical trace and the second electrical trace when the first electrical trace and the second electrical trace are in contact with the body of the user.

Clause 20: The device of claim 17, wherein the housing comprises a rim, and wherein a portion of the plurality of electrical traces is positioned on the rim of the housing.

### BRIEF DESCRIPTION OF THE DRAWINGS

Additional advantages and details are explained in greater detail below with reference to the exemplary embodiments that are illustrated in the accompanying schematic figures, in which:

FIG. 1 is a perspective view of a non-limiting embodiment of a disinfection device;

FIG. 2 is an exploded view of the disinfection device shown in FIG. 1;

FIG. 3 is a cross-sectional view of the disinfection device shown in FIG. 1 along line A-A of FIG. 1;

FIG. 4 a cross-sectional view of the housing shown in FIG. 3 along line B-B of FIG. 2;

FIG. 5 is a side view of a non-limiting embodiment of a housing of the disinfection device shown in FIG. 2;

FIG. 6 is a perspective view of a non-limiting embodiment of a band; and

FIG. 7 is a perspective view of the disinfection device shown in FIG. 1 positioned within the band shown in FIG. 6.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods, and products for disinfecting a surface using a light source. Embodiments of the present disclosure may include a disinfection device that includes a housing having a concave shape, a light source coupled to the housing, where the light source provides light configured to disinfect a surface, and a contact sensor coupled to the housing, where the contact sensor is configured to detect contact with a body of a user. In some non-limiting embodiments, the light source provides light in a wavelength that is configured to disinfect a surface. In some non-limiting embodiments, the light source includes at least one light emitting diode (LED). In some non-limiting embodiments, the light source provides antimicrobial blue light (aBL). In some non-limiting embodiments, the device includes a printed circuit board (PCB) positioned within the housing and the light source is attached to the PCB. In some non-limiting embodiments, the contact sensor includes a plurality of electrical traces. In some non-limiting embodiments, the contact sensor comprises a sensor in electrical contact with the plurality of electrical traces. In some non-limiting embodiments, the contact sensor includes a first electrical trace and a second electrical trace, wherein the first electrical trace and the second electrical trace are separated by a gap. In some non-limiting embodiments, the contact sensor is configured to detect contact with the body of the user based on a reduction in resistance between the first electrical trace and the second electrical trace when the first electrical trace and the second electrical trace are in contact with the body of the user. In some non-limiting embodiments, the housing includes a rim, and the contact sensor is positioned on the rim of the housing. In some non-limiting embodiments, the light source is configured to be activated based on the contact sensor detecting contact with the body of the user. In some non-limiting embodiments, the device includes a band configured to hold the housing in contact with the body of the user. In some non-limiting embodiments, the device includes a proximity sensor attached to the PCB, where the proximity sensor is configured to detect whether the proximity sensor is in proximity to the body of the user.

In this way, embodiments of the present disclosure provide a device for disinfecting intravenous (IV) injection sites, including IV catheter insertion sites, without disturbing any devices used at the IV injection sites, such as an IV dressing that holds an IV line in place.

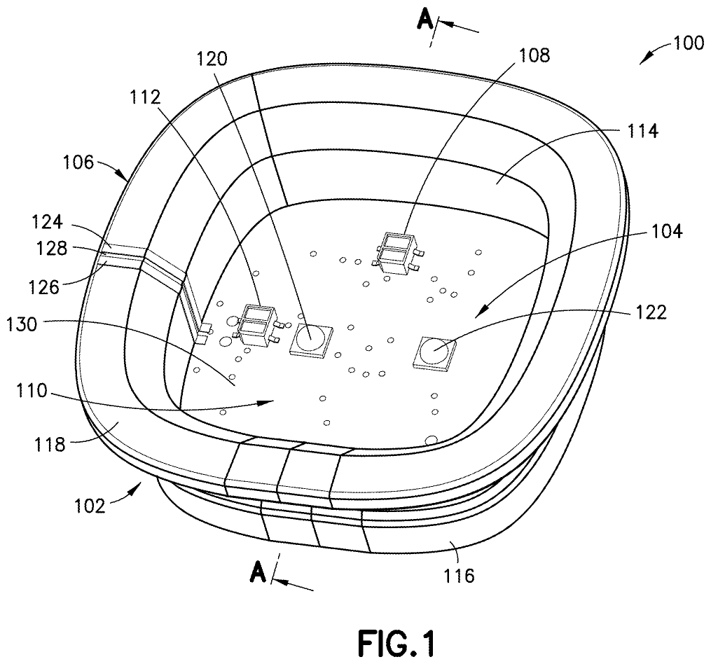

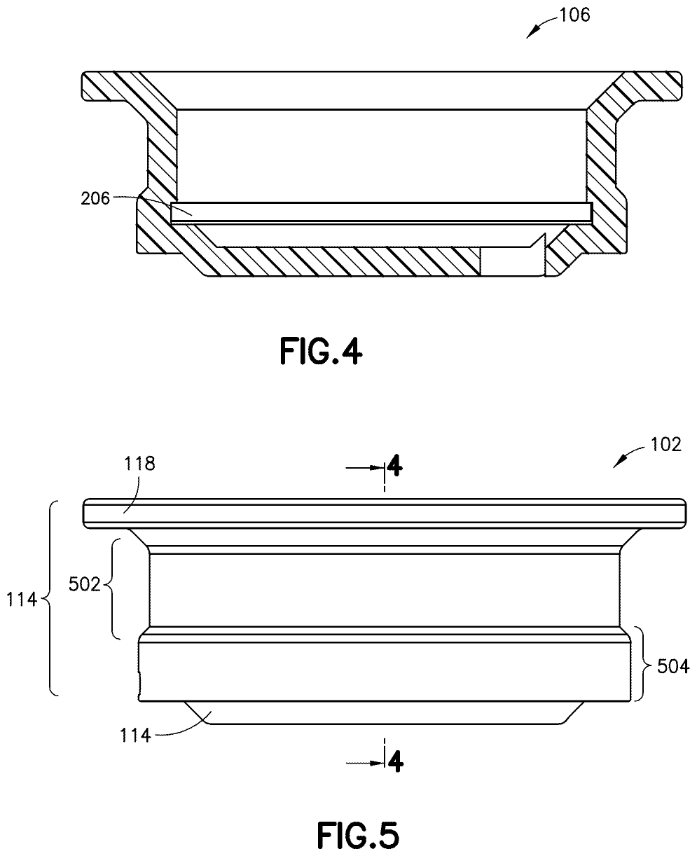

Referring now to FIGS. 1-4, FIGS. 1-4 are diagrams of disinfection device 100 and components thereof. As shown in FIGS. 1-3, disinfection device 100 may include housing 102, light source 104, contact sensor 106, proximity sensor 108, printed circuit board (PCB) 110, and controller 112. In some non-limiting embodiments, housing 102 of disinfection device 100 may have a concave shape. In this way, disinfection device 100 may be used to cover an IV injection site, such as an IV catheter insertion site, to provide a barrier to contamination of the IV injection site as well as providing light (e.g., in the form of electromagnetic radiation) that is configured to disinfect a surface covered by disinfection device 100. In some non-limiting embodiments, housing 102 may include side wall section 114 that is coupled to (e.g., formed on) base section 116 such that housing 102 has a concave shape. In some non-limiting embodiments, housing 102 may include rim 118. For example, housing 102 may include rim 118 that is coupled to side wall section 114.

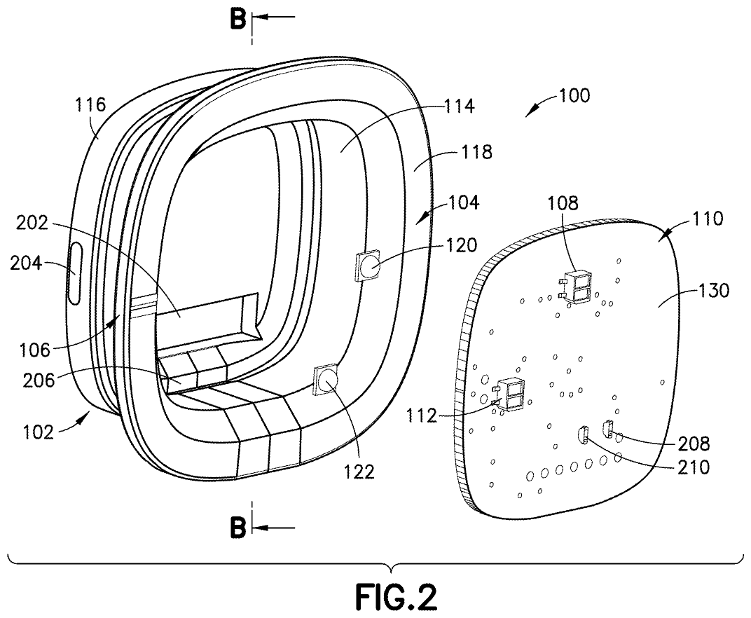

In some non-limiting embodiments, housing 102 may include (e.g., may be constructed of) a flexible material so that housing 102 may conform to a surface to which disinfection device 100 may be applied. In some non-limiting embodiments, the hardness of a material from which housing 102 may be based on an area of an application of housing 102 on the body of a patient. For example, the material may softer if housing 102 is to be applied to a hand or neck of a patient and the material may be harder for relatively flat body part, such as a chest. The hardness of the material of housing 102 can be in the range of 25 Shore A to 50 Shore D. In one example, the range may be 30 Shore A to 90 Shore A. In another example, the range of 35 Shore A may be 70 Shore A. In some non-limiting embodiments, the material from which housing 102 is constructed may include a polymer, such as, but not limited to, a plastic (e.g., polypropylene or polyethylene), a thermoplastic elastomer (e.g., styrene block copolymer, butyl, thermoplastic olefin, and/or thermoplastic urethanes), and/or rubber (e.g., polyisoprene, thermoplastic vulcanizates, etc.). In some non-limiting embodiments, the material from which housing 102 is constructed may exclude natural rubber. This may help to prevent allergic reactions in patients that have a latex allergy. Additionally or alternatively, housing 102 may include a flexible polymeric foam. In some non-limiting embodiments, housing 102 may be formed as a unitary component (e.g., as a single piece). For example, housing 102 may be molded (e.g., injection molded) as a unitary component. As shown in FIGS. 2-4, housing 102 may include first aperture 202, second aperture 204, and channel 206. In some non-limiting embodiments, first aperture 202 may be defined in (e.g., formed in, cutout from, etc.) base section 114 of housing 102. First aperture 202 may be sized and configured to allow a user to view and/or interact with electronic components positioned on a rearward surface (e.g. a surface of PCB 110 opposite front surface 130 of PCB 110) of PCB 110. As shown in FIG. 2, first aperture 202 may have a rectangular shape. In some non-limiting embodiments, second aperture 204 may be defined in (e.g., formed in, cutout from, etc.) side wall section 114 of housing 102. Second aperture 204 may be sized and configured to allow a user to interface (e.g., receive device information, provide control settings, adjust program parameters, etc.) with an electronic component (e.g., controller 112, light source 104, proximity sensor 108, etc.) on PCB 110. For example, second aperture 204 may be sized and configured to allow a user to insert a connector through second aperture 204 into a socket (e.g., a plug) of the electronic component to interface with the electronic component. As shown in FIG. 2, second aperture 204 may have a rounded rectangle shape. In some non-limiting embodiments, channel 206 may be defined in (e.g., formed in, cutout from, etc.) an interior surface of side wall section 114 of housing 102. Channel 206 may be sized and configured to accommodate PCB 110, when PCB 110 is positioned within housing. For example, channel 206 may be sized and configured to allow PCB 110 to fit within channel 206 based on a thickness (e.g., a height) of PCB 110. As shown in FIG. 2, channel 206 may be defined annularly along the interior surface of side wall section 114, adjacent base section 116 of housing 102.

In some non-limiting embodiments, light source 104 may include a device that is configured to provide light that is configured to disinfect a surface. For example, light source 104 may include a device that is configured to provide light in a wavelength that is configured to disinfect a surface. In this way, disinfection device 100 may provide for disinfection (e.g., antimicrobial disinfection) of a surface (e.g., a surface of an IV injection site), such as an appendage (e.g., an arm and/or a leg) of a user based on the use of light source 104. In addition, disinfection device 100 may reduce phlebitis at site of a user on which disinfection device 100 is applied. In some non-limiting embodiments, light source 104 may include a light emitting diode (LED), a plurality of LEDs (e.g., an array of LEDs), a light bulb, a light-emitting electrochemical cell (LEC), a laser, and/or the like.

In some non-limiting embodiments, light source 104 may provide antimicrobial blue light (aBL), which may include electromagnetic radiation having a wavelength between 400-470 nm. Additionally or alternatively, light source 104 may provide electromagnetic radiation having a wavelength between 630-680 nm, which may be referred to as red light. Additionally or alternatively, light source 104 may provide electromagnetic radiation having a wavelength between 10-400 nm, which may include electromagnetic radiation in the ultraviolet (UV) spectrum. For example, light source 104 may provide electromagnetic radiation having a wavelength between 100-280 nm (e.g., the UVC spectrum), electromagnetic radiation having a wavelength between 280-315 nm (e.g., the UVB spectrum), and/or electromagnetic radiation having a wavelength between 315-400 nm (e.g., the UVA spectrum).

In some non-limiting embodiments, light source 104 may be coupled to housing 102. For example, light source 104 may be attached to housing 102. As shown in FIGS. 1-2, light source 104 may include LEDs 120, 122. LEDs 120, 122 may be coupled to PCB 110 and PCB 110 may be coupled to housing 102. For example, LEDs 120, 122 may be attached (e.g., attached via a solder joint) to PCB 110, which is coupled to (e.g., attached to, positioned within, etc.) housing 102. In some non-limiting embodiments, LED 120 and LED 122 may include LEDs that are of different colors. For example, LED 120 may include a red LED and LED 122 may include a blue LED, or vice versa. In some non-limiting embodiments, LED 120 and LED 122 may include LEDs that are of the same color. For example, LED 120 may include a blue LED and LED 122 may include a blue LED, or LED 120 may include a red LED and LED 122 may include a red LED. In some non-limiting embodiments, LED 120 and/or 122 may include a high brightness (e.g., high intensity) LED.

In some non-limiting embodiments, contact sensor 106 may be coupled to housing 102. For example, contact sensor 106 may be positioned on rim 118 of housing 102. In some non-limiting embodiments, contact sensor 106 may include a device (e.g., a sensor) configured to detect contact of disinfection device 100 with a surface, such as a body (e.g., an appendage of a body) of a user. In some non-limiting embodiments, contact sensor 106 may include a plurality of electrical traces. In some non-limiting embodiments, contact sensor 106 may include a sensor (e.g., a sensor of controller 112) in electrical contact with the plurality of electrical traces and the sensor may be configured to determine whether a conductive path is present from a first electrical trace of the plurality of electrical traces to a second electrical trace of the plurality of electrical traces.

As shown in FIG. 1, contact sensor 106 may include first electrical trace 124 and second electrical trace 126, where first electrical trace 124 and second electrical trace 126 are separated by gap 128. In some non-limiting embodiments, contact sensor 106 may detect contact with the body of the user based on a reduction in resistance between first electrical trace 124 and second electrical trace 126 when first electrical trace 124 and second electrical trace 126 are in contact with the body of the user. In this way, contact sensor 106 may detect contact with the body of the user based on the body of the user providing a conductive path over gap 128 between first electrical trace 124 and second electrical trace 126. In some non-limiting embodiments, gap 128 may be about 0.01 inches (e.g., 0.01 inches ±10% based on manufacturing tolerances). In some non-limiting embodiments, gap 128 may be sized and configured to optimize the resistance of the skin of a patient. As further shown in FIG. 1, a first end of first electrical trace 124 and a first end of second electrical trace 126 may be positioned on rim 118 of housing 102. First electrical trace 124 and second electrical trace 126 may be positioned on (e.g., positioned to run along) an interior surface of side wall section 114 and a second end of first electrical trace 124 and a second end of second electrical trace 126 may be positioned on PCB 110. In some non-limiting embodiments, the second end of first electrical trace 124 and the second end of second electrical trace 126 may be in electrical contact with controller 112.

In some non-limiting embodiments, proximity sensor 108 may include a device (e.g., a sensor) configured to detect whether the device is in proximity (e.g., when disinfection device 100 satisfies a condition of being near) to a surface, such as a body (e.g., an appendage of a body) of a user. In some non-limiting embodiments, proximity sensor 108 may include a device configured to detect when disinfection device 100 is within a predetermined threshold of distance to the surface. As shown in FIGS. 1 and 2, proximity sensor 108 may be coupled to (e.g., attached to via a solder joint) PCB 110. In some non-limiting embodiments, disinfection device 100 may include only one of contact sensor 106 or proximity sensor 108. For example, disinfection device 100 may include contact sensor 106 and not include proximity sensor 108, or vice versa. In some non-limiting embodiments, disinfection device 100 may include both contact sensor 106 and proximity sensor 108. For example, disinfection device 100 may include contact sensor 106 and proximity sensor 108, where contact sensor 106 may be configured to act as a backup sensor for proximity sensor 108 or proximity sensor 108 may be configured to act as a backup sensor for contact sensor 106. In some non-limiting embodiments, where contact sensor 106 detects contact with a body of a patient and proximity sensor 108 does not detect the body of the patient in proximity to proximity sensor 108 or where contact sensor 106 does not detect contact with a body of a patient and proximity sensor 108 detects the body of the patient in proximity to proximity sensor 108 disinfection device 100 may enter into an operational state (e.g., a safe state) in which light source 104 is disabled. In some non-limiting embodiments, disinfection device 100 may include a thermal sensor (e.g., a non-contact thermal sensor). In some non-limiting embodiments, the thermal sensor may be configured to operate as a failsafe to shut down or modulate operation of light source 104, if light source 104 causes the temperature (e.g., the temperature as detected by the thermal sensor) of a dressing (e.g., an IV catheter dressing, wound dressing, etc.) and/or skin of the patient to an unsafe level.

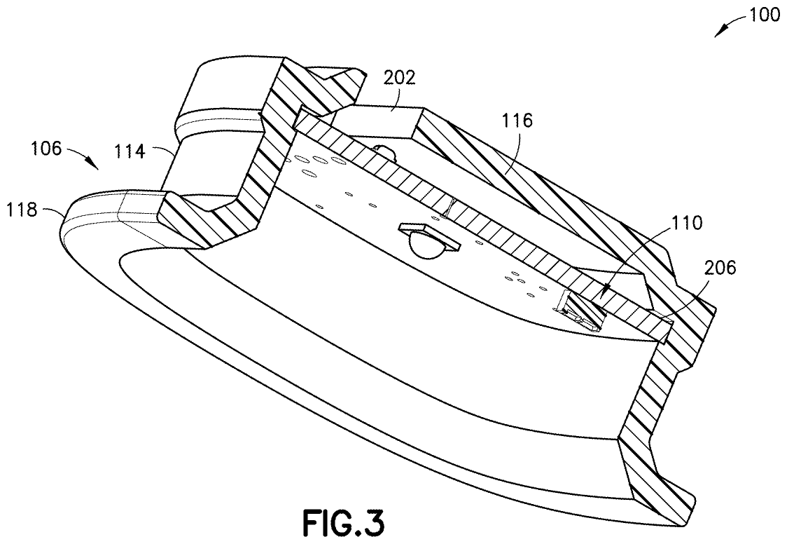

As shown in FIG. 3, PCB 110 may be positioned within channel 206 of housing 102. In some non-limiting embodiments, PCB 110 may include a plurality of electrical components attached thereto. For example, PCB 110 may include light source 104 (e.g., LEDs 120, 122 of light source 104), contact sensor 106 (e.g., a portion of electrical traces 124, 126 or contact sensor 106, proximity sensor 108, controller 112, and/or status indicator LEDs 208, 210 attached to front surface 130 of PCB 110. In some non-limiting embodiments, one or more of light source 104 (e.g., LEDs 120, 122 of light source 104), contact sensor 106 (e.g., a portion of electrical traces 124, 126 or contact sensor 106, proximity sensor 108, controller 112, and/or status indicator LEDs 208, 210 may be attached to a rear surface of PCB 110. In some non-limiting embodiments, one or more of LEDs 120, 122 of light source 104 may be positioned within (e.g., such that a portion of one or more of LEDs 120, 122 extends through) one or more apertures in PCB 110. In some non-limiting embodiments, the plurality of electrical components may be coupled to PCB 110 in any appropriate way. For example, the plurality of electrical components may be attached (e.g., attached via solder joints), formed on, deposited on, and/or the like.

In some non-limiting embodiments, controller 112 may include a device configured to control light source 104. For example, controller 112 may include a device configured to control (e.g., control the activation of) light source 104 based on information received from contact sensor 106 and/or proximity sensor 108. In some non-limiting embodiments, controller 112 may include a processor, such as a central processing unit (CPU), a microcontroller, an integrated circuit (IC), and/or the like. In some non-limiting embodiments, controller 112 may control light source 104 (e.g., may control LEDs 120, 122 simultaneously, such as in combination, or may control LEDs 120, 122 separately, such as in a sequential manner) by providing power to light source 104 from a power source. For example, controller 112 may control light source 104 based on data stored in a memory (e.g., data associated with a pre-programmed disinfecting time) associated with controller 112 providing power to light source 104 from the power source. In some non-limiting embodiments, controller 112 may control a circuit or components of a circuit to provide power to light source 104 from the power source. For example, controller 112 may control a transistor to provide power to light source 104 from the power source.

In some non-limiting embodiments, controller 112 may control light source 104 based on a predetermined time interval (e.g., a predetermined duration of time, a predetermined time period, a predetermined amount of time, etc.), which may be associated with a time for disinfecting a surface. For example, controller 112 may activate light source 104 for the predetermined time interval and upon the expiration of the predetermined time interval, controller 112 may deactivate light source 104. In some non-limiting embodiments, the predetermined time interval may be greater than or equal to 90 minutes. In some non-limiting embodiments, light source 104 is configured to be activated based on contact sensor 106 detecting that disinfection device 100 is in contact with a body of a user and/or proximity sensor 108 detecting that disinfection device 100 is in proximity to the body of the user. For example, controller 112 may activate light source 104 based on receiving information from contact sensor 106 that indicates that disinfection device 100 is in contact with a body of a user and/or receiving information from proximity sensor 108 indicating that disinfection device 100 is in proximity to the body of the user.

In some non-limiting embodiments, controller 112 may control light source 104 based on information (e.g., data, such as a signal) received from contact sensor 106 and/or proximity sensor 108. For example, controller 112 may activate light source 104 based on receiving information from contact sensor 106 that indicates disinfection device 100 is in contact with a body of a user. Additionally or alternatively, controller 112 may activate light source 104 based on receiving information from proximity sensor 108 that indicates disinfection device 100 (e.g., proximity sensor 108 of disinfection device 100) is in proximity to (e.g., is within a predetermined threshold of distance to) the surface the body of the user. In some non-limiting embodiments, controller 112 may deactivate light source 104 based on receiving information from contact sensor 106 that indicates disinfection device 100 is not in contact with the body of the user. Additionally or alternatively, controller 112 may deactivate light source 104 based on receiving information from proximity sensor 108 that indicates disinfection device 100 is not in proximity to (e.g., is not within a predetermined threshold of distance to) the surface a body of a user. In this way, contact sensor 106 and/or proximity sensor 108 may prevent undesirable activation of light source 104, for example, to prevent light from light source 104 from being shone into the eyes of the user or another individual. Additionally or alternatively, controller 112 may control light source 104 based on a user input. For example, controller 112 may control light source 104 based on receiving a user input via an interface component (e.g., a button, such as a start button or stop button).

In some non-limiting embodiments, controller 112 may provide an indication of a status of disinfection device 100. For example, controller 112 may control status indicator LEDs 208, 210 to provide an indication of a status of disinfection device 100. In some non-limiting embodiments, status indicator LEDs 208, 210 may include LEDs that are of different colors. For example, status indicator LED 208 may include a red LED and status indicator LED 210 may include a green LED, or vice versa. In some non-limiting embodiments, status indicator LEDs 208, 210 may include LEDs that are of the same color. For example, status indicator LED 208 may include a green LED and status indicator LED 210 may include a green LED, or status indicator LED 208 may include a red LED and status indicator LED 210 may include a red LED. In some non-limiting embodiments, status indicator LEDs 208, 210 may include a high brightness (e.g., high intensity) LED.

In some non-limiting embodiments, controller 112 may activate one or more of status indicator LEDs 208, 210 to provide an indication of a status of disinfection device 100 based on the expiration of a predetermined time interval during which one or more of LEDs 120, 122 had been activated to disinfect a surface. For example, controller 112 may activate one or more of LEDs 120, 122 during the predetermined time interval to disinfect an IV injection site upon which disinfection device 100 has been positioned. Controller 112 may determine that the predetermined time interval has expired, and controller 112 may deactivate one or more of LEDs 120, 122 based on determining that the predetermined time interval has expired. Controller 112 may activate one or more of control status indicator LEDs 208, 210 to provide an indication that a disinfection procedure involving disinfection device 100 has been completed based on deactivating one or more of LEDs 120, 122.

Referring now to FIG. 5, FIG. 5 is a diagram of housing 102. As shown in FIG. 5, side wall section 114 of housing 102 may include upper side wall section 502 and lower side wall section 504. In some non-limiting embodiments, lower side wall section 504 may be formed above base section 116 and lower side wall section 504 may be wider than base section 116 at a location where lower side wall section 504 meets base section 116. In some non-limiting embodiments, lower side wall section 504 may be formed below upper side wall section 502, and lower side wall section 504 may taper to a location at which lower side wall section 504 meets upper side wall section 502, such that a width of lower side wall section 504 at the location where lower side wall section 504 meets upper side wall section 502 is less than a maximum width of lower side wall section 504. In some non-limiting embodiments, upper side wall section 502 may be formed above lower side wall section 504, and upper side wall section 502 may taper to a location at which upper side wall section 502 meets rim 118, such that a width of upper side wall section 502 at the location where upper side wall section 502 meets rim 118 is greater than a width of upper side wall section 502 at the location where upper side wall section 502 meets lower side wall section 504. In this way, side wall section 114 of housing 102 provides for the ability of housing 102 to be securely held against a surface, such as a body of a user, while still providing an appropriate distance between the surface and light source 104 of disinfection device 100. In some non-limiting embodiments, a width of housing 12 may be 95% of a width of an IV catheter dressing. In some non-limiting embodiments, a height of housing 102 may be about 0.5 inches (e.g., 0.5 inches ±10% based on manufacturing tolerances). In some non-limiting embodiments, side wall section 114 of housing 102 may be sized and configured to provide a distance (e.g., a standoff distance, a height, etc.) between light source 104 and a location (e.g., a location of an IV injection site, a location of an IV dressing, a location of the surface of the skin of a patient, etc.) at which disinfection device 100 is to be applied. In this way, side wall section 114 of housing 102 may ensure that light source 104 does not directly contact the location, as to avoid unsafe thermal heating.

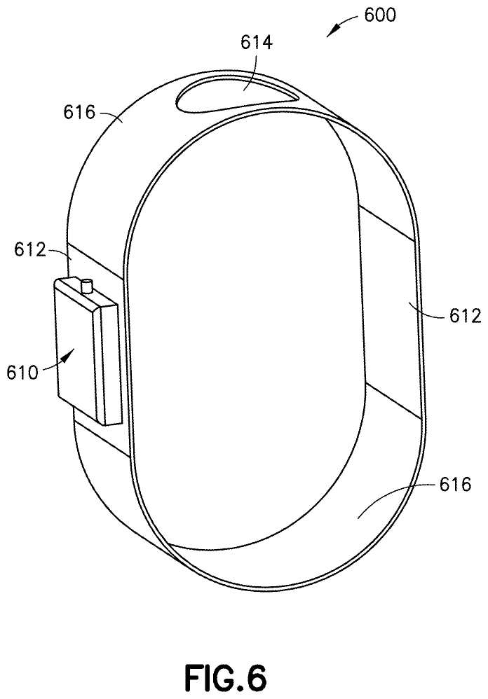

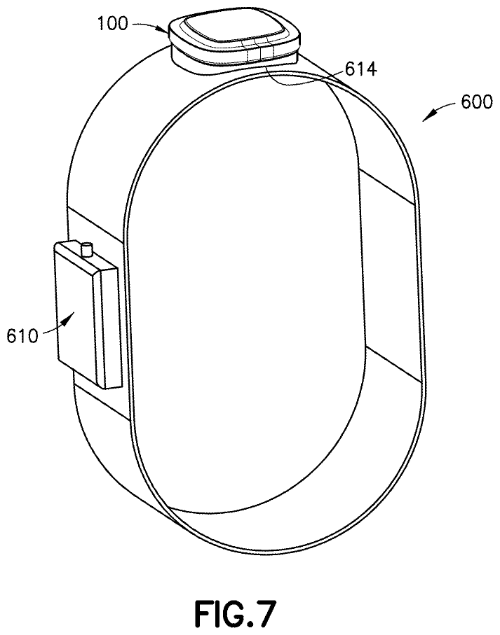

Referring now to FIG. 6, FIG. 6 is a diagram of band 600. As shown in FIG. 6, band 600 may include flexible sections 616, inflexible sections 612, and aperture 614. In some non-limiting embodiments, aperture 614 is sized and configured to hold disinfection device 100 (e.g., as shown in FIG. 7). As further shown in FIG. 6, band 600 may include power component 610 attached to inflexible section 612. Power component 610 may include a power source that is used to provide power to disinfection device 100. In some non-limiting embodiments, power component 610 may include a power source that is used to charge a power source of disinfection device 100. For example, power component 610 may include a power source that is used to charge the power source of disinfection device 100 via wireless induction.

In some non-limiting embodiments, band 600 may be sized and configured to fit a specific area of a body of a user. For example, band 600 may be sized and configured to fit on an arm of the user. In some non-limiting embodiments, band 600 may be formed from a material that is appropriate for allowing band 600 to hold disinfection device 100 in a position on the body of the user. In some non-limiting embodiments, flexible sections 616 of band 600 may be formed from a material that stretches, such as elastic and/or neoprene, and inflexible sections 612 of band 600 may be formed from a material that does not stretch as much as the material of flexible sections 616, such as cotton and/or polyester. In some non-limiting embodiments, band 600 may be formed from a single material (e.g., band 600 does not include flexible sections 616 and inflexible sections 612 of different material, and instead band 600 is formed from one type of material). In some non-limiting embodiments, band 600 may be formed as a loop, such that band 600 may be placed around an appendage of a user by inserting the appendage through band 600. In some non-limiting embodiments, band 600 may include a fastening element, such as a hook and loop fastening element, a button, a belt buckle, and/or the like, so that band 600 may be placed around a body or an appendage of a body a user by wrapping band 600 around the body or the appendage and securing band 600 using the fastening element.

Referring now to FIG. 7, FIG. 7 is a diagram of band 600 and disinfection device 100. As shown in FIG. 7, disinfection device 100 may be positioned within aperture 614 of band 600, and within an appropriate distance of power component 610. During use, a user or an administrator of disinfection device 100 may apply disinfection device 100 to a location (e.g., a location of an IV injection site) on a body of the user via band 600 and initiate a disinfection procedure. During the disinfection procedure, band 600 is configured to hold disinfection device 100 in place while disinfection device 100 covers the location without disturbing any devices that are positioned underneath disinfection device 100, such as an IV catheter. Once, the disinfection procedure is complete, the user or the administrator may remove disinfection device 100 from the location on the body of the user via band 600 without disturbing any devices that are positioned underneath disinfection device 100.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A disinfection device comprising:
a housing having a concave shape;
a light source coupled to the housing, wherein the light source provides light configured to disinfect a surface;
a contact sensor coupled to the housing; and
wherein the contact sensor is configured to detect contact with a body of a user.

2. The disinfection device of claim 1, wherein the light source provides light in a wavelength that is configured to disinfect a surface.

3. The disinfection device of claim 2, wherein the light source comprises at least one light emitting diode (LED).

4. The disinfection device of claim 2, wherein the light source provides antimicrobial blue light (aBL).

5. The disinfection device of claim 1, further comprising:
a printed circuit board (PCB) positioned within the housing; and
wherein the light source is attached to the PCB.

6. The disinfection device of claim 1, wherein the contact sensor comprises a plurality of electrical traces.

7. The disinfection device of claim 6, wherein the contact sensor comprises a sensor in electrical contact with the plurality of electrical traces.

8. The disinfection device of claim 1, wherein the contact sensor comprises a first electrical trace and a second electrical trace, wherein the first electrical trace and the second electrical trace are separated by a gap.

9. The disinfection device of claim 8, wherein the contact sensor is configured to detect contact with the body of the user based on a reduction in resistance between the first electrical trace and the second electrical trace when the first electrical trace and the second electrical trace are in contact with the body of the user.

10. The disinfection device of claim 1, wherein the housing comprises a rim, and wherein the contact sensor is positioned on the rim of the housing.

11. The disinfection device of claim 1, wherein the light source is configured to be activated based on the contact sensor detecting contact with the body of the user.

12. The disinfection device of claim 1, further comprising:
a band configured to hold the housing in contact with the body of the user.

13. A device comprising:
a housing having a concave shape;
a printed circuit board (PCB) positioned within the housing;
a light source attached to the PCB, wherein the light source provides light configured to disinfect a surface;
a contact sensor coupled to the housing, wherein the contact sensor is configured to detect contact with a body of a user; and
a proximity sensor attached to the PCB, wherein the proximity sensor is configured to detect whether the proximity sensor is in proximity to the body of the user.

14. The device of claim 13, wherein the light source comprises at least one light emitting diode (LED) that provides light in a wavelength that is configured to disinfect a surface.

15. The device of claim 14, wherein the at least one LED provides antimicrobial blue light (aBL).

16. The device of claim 13, wherein the contact sensor comprises a first electrical trace and a second electrical trace, wherein the first electrical trace and the second electrical trace are separated by a gap; and
wherein the contact sensor is configured to detect contact with the body of the user based on a reduction in resistance between the first electrical trace and the second electrical trace when the first electrical trace and the second electrical trace are in contact with the body of the user.

17. A device comprising:
a housing having a concave shape;
a printed circuit board (PCB) positioned within the housing;
a light source attached to the PCB, wherein the light source provides light configured to disinfect a surface; and
a contact sensor comprising a plurality of electrical traces and a sensor electrically coupled to the plurality of electrical traces, wherein the sensor is attached to the PCB; and
wherein the contact sensor is configured to detect contact with a body of a user.

18. The device of claim 17, wherein the light source comprises at least one light emitting diode (LED) that provides antimicrobial blue light (aBL).

19. The device of claim 17, wherein the contact sensor comprises a first electrical trace and a second electrical trace, wherein the first electrical trace and the second electrical trace are separated by a gap; and
wherein the contact sensor is configured to detect contact with the body of the user based on a reduction in resistance between the first electrical trace and the second electrical trace when the first electrical trace and the second electrical trace are in contact with the body of the user.

20. The device of claim 17, wherein the housing comprises a rim, and wherein a portion of the plurality of electrical traces is positioned on the rim of the housing.
